(19) United States
(12) Patent Application Publication
(10) Pub. No.: US 2026/0174322 A1
(43) Pub. Date: Jun. 25, 2026

(54) ENDOSCOPE DEVICE AND ENDOSCOPE WITH AN ENDOSCOPE DEVICE

(71) Applicant: KARL STORZ SE & Co. KG, Tuttlingen (DE)

(72) Inventor: Peter SCHWARZ, Tuttlingen (DE)

(21) Appl. No.: 19/425,375

(22) Filed: Dec. 18, 2025

(30) Foreign Application Priority Data
Dec. 19, 2024 (DE) 10 2024 138 870.6

(51) Int. Cl. A61B 1/313; A61B 1/00; A61B 1/005; A61B 1/05; A61B 1/12

(52) U.S. Cl. CPC A61B 1/3132; A61B 1/00114; A61B 1/00117; A61B 1/00119; A61B 1/00128; A61B 1/00137; A61B 1/00179; A61B 1/00193; A61B 1/0055; A61B 1/05; A61B 1/126; A61B 2560/0462

(57) ABSTRACT

The invention relates to an endoscope device (10) comprising: a shaft (20) with a shaft longitudinal axis (30); a camera receptacle (40); at least one linking element (50) to which the camera receptacle (40) is movably attached; a camera module (60) that has a stereoscopic image recording unit (70) and which can be accommodated in the camera receptacle (40), wherein a mobility of the camera receptacle (40) can be released relative to the linking element (50) by displacing the camera receptacle (40) and the linking element (50) relative to the shaft (20), thereby allowing the camera module (60) to be deflected from an insertion position to an image capturing position, wherein in the image capturing position, the camera module (60) defines a viewing direction that is at an angle relative to the shaft longitudinal axis, and wherein in the image capturing position, the image recording unit (70) is rotatably mounted to rectify the image relative to the camera receptacle (40).

The invention further relates to an endoscope with an endoscope device according to the invention.

The present invention relates to an endoscope device, in particular a stereoscopic laparoscope, and to a system with an endoscope device.

In minimally invasive surgery, endoscopes form part of the prior art, enabling the creation of magnified images of a region within a patient's body that is under examination. For improved visualization, stereoscopic image recording units are used in endoscopes, which can visualize the environment to a surgeon and/or another user in 3-D and/or 2-D.

To give the surgeon and/or other user a good overview, the image recording units are usually angled toward a longitudinal axis of the endoscope in order to visualize images at an angle to the longitudinal axis of the endoscope and thus in a so-called oblique view. This usually involves the use of endoscopes that have a 30°, and/or 45°, and/or 60°, and/or 75° oblique view.

When designing endoscopes with a stereoscopic image recording unit that are intended to have an oblique view during use, it is especially difficult in particular to reduce the outer diameters in such a way that the minimally invasive procedure can be kept as small as possible. Furthermore, to improve user orientation in 3-D and/or 2-D representations, the image from the image recording unit is to be rectified, if necessary. While 2-D representations can also be rectified electronically, this is not possible with 3-D representations. Image rectification can be achieved, for example, through complex mechanical systems for moving the individual image sensors.

Furthermore, sufficient light must be provided at the distal end of the endoscope to adequately illuminate the inside of the patient's body in order to record the best possible images.

In addition, the medical imaging capability of endoscopes allow different types of tissue, such as organs and/or blood vessels and/or tumor tissue, to be visualized at different depths under the skin or in the body cavity. Fluorescence imaging methods are used to be able to better differentiate the tissue types. For this purpose, the patient is given a drug containing a fluorescent dye, in particular fluorophores, which is deposited in one of the different types of tissue. Particularly with these fluorescence imaging methods, sufficient excitation light must be provided at the distal end of the endoscope in order to be able to adequately excite the fluorescent substances.

In known endoscopes with an oblique view, distal prisms are used to achieve the oblique view. Especially with oblique-view endoscopes, the integration of sufficient illumination fibers presents a significant challenge, since the distal prisms severely restrict the space within the endoscope for integrating illumination fibers.

Furthermore, it is important to be able to flush the distal end of the endoscope during use. A fluid is usually used to flush the distal end of the endoscope and is guided to the distal end of the endoscope via corresponding lines. However, integrating the lines into known endoscopes sometimes requires an additional flushing shaft, which must also be attached to the endoscope. The additional shaft unnecessarily increases the outer diameter of the endoscope. Therefore, due to the additional shaft, a trocar must be used, which has a larger diameter and unnecessarily enlarges the minimally invasive procedure.

DE10004264C2 discloses a stereo camera that can be spread apart from the shaft of an endoscope via joints. By advancing an instrument, the spreading is controlled, and the instrument is kept within the camera's field of view.

WO2014104405A1 describes an endoscope with a stereo camera that can be positioned at different angles and can be moved into the angled position by a thrust mechanism.

A similar mechanism is also known from EP2123225A1.

U.S. Pat. No. 11,064,867B2 discloses an endoscope, wherein a distal camera unit is deflected outward by advancing a hollow shaft within the instrument and is thus moved into a position that is laterally offset relative to the shaft.

Proceeding from the prior art, the object addressed by the invention is in particular that of creating an endoscope with an oblique view that enables a user to intuitively rectify an image, but is not limited thereto.

Furthermore, proceeding from the prior art, the object addressed by the invention is in particular that of providing, with the smallest possible minimally invasive procedure, plenty of space, e.g., for the integration of lighting fibers and/or fluid lines, but is not limited thereto.

At least one of these objects is achieved according to the invention by an endoscope device and by a endoscope with an endoscope device, as are described herein and defined in the claims.

The present invention provides an endoscope device with a shaft having a shaft longitudinal axis. The endoscope device further comprises a camera receptacle, at least one linking element to which the camera receptacle is movably attached, and a camera module which has a stereoscopic image recording unit and can be accommodated in the camera receptacle. Furthermore, in the endoscope device according to the invention, in particular articulated mobility of the camera receptacle relative to the linking element can be enabled by moving the camera receptacle and the linking element relative to the shaft, whereby the camera module can be deflected from an insertion position to an image capturing position. Furthermore, in the image capturing position, the camera module defines a viewing direction that is angled relative to the longitudinal shaft axis. In addition, in the image capturing position, the image recording unit is mounted so that it can rotate relative to the camera receptacle in order to align the image.

The movement made possible by the release of the mobility is, or includes in particular, a deflection.

In particular, the camera module and/or the camera receptacle and/or the at least one linking element can be accommodated in an insertion position in the shaft. In particular, the endoscope device is inserted inside the patient's body in the insertion position. For example, the endoscope device can be inserted inside the body through a trocar. In the image capturing position, the endoscope device in particular has an oblique view. The oblique view can, for example, be at an angle of 30° or 45° or 60° or 75°. For example, in the image capturing position, the endoscope device can be designed to record an image of an environment, such as the inside of the patient's body.

In particular, the shaft extends from a proximal end to a distal end of the endoscope device. In particular, the shaft is rigid and/or flexible. In particular, the shaft is round and/or angular. Preferably, the shaft is made entirely or partially of metal, ceramic, and/or plastic. The longitudinal axis of the shaft extends in particular from the proximal end to the distal end of the shaft. In particular, the longitudinal shaft axis passes through the center point of a cross-section of the shaft.

The camera receptacle is in particular designed to partially or completely accommodate the camera module in the image capturing position. The camera receptacle can be designed articulated or can be in particular designed to release movement between the camera receptacle and the linking element. In other words, the camera receptacle is movable with respect to the linking element.

The camera module and/or the camera receptacle and/or at least one linking element can have a similar outer diameter. In particular, the camera module and/or the camera receptacle and/or the at least one linking element has an outer diameter that is flush with the inner diameter of the shaft and/or is designed in such a way that the camera module and/or the camera receptacle and/or the at least one linking element can be inserted into the shaft. However, the camera module and/or the camera receptacle can also have an outer diameter that is larger than the inner diameter of the shaft. In particular, the outer diameter of the camera receptacle and/or camera module can have a similar and/or comparable and/or identical outer diameter to the shaft.

In particular, to achieve the articulated mobility between the camera receptacle and the at least one linking element and/or the linking elements, a connection between the camera receptacle and the linking element and/or a connection between the linking elements can have a slot. Otherwise, the connection between the camera receptacle and the linking element can, in particular, have a constant cross-section. Alternatively and/or additionally, the camera receptacle and the linking element and/or the linking elements can be connected via a joint, for example a hinge joint and/or a solid body joint. The linking elements can be permanently or detachably connected to each other.

In particular, due to the articulated mobility between the camera receptacle and the linking element, the image capturing position can be deflected at a first angle with respect to the longitudinal shaft axis. Due to the in particular articulated mobility between two linking elements, the image capturing position can in particular be deflected at a second angle with respect to the longitudinal shaft axis. That is, if the in particular articulated mobility of the camera receptacle is released, or if the articulated mobility between the camera receptacle and the linking element to which the camera receptacle is connected is released, the angle of the image recording position with respect to the shaft longitudinal axis corresponds to the first angle. If, in addition, the in particular articulated mobility of the linking element to which the camera receptacle is connected is released for a further linking element, the angle of the image capturing position with respect to the shaft longitudinal axis corresponds to the second angle. If, in addition, the in particular articulated mobility of the further linking element is released, the angle of the image capturing position with respect to the shaft longitudinal axis corresponds to a further angle. In particular, the further angle and/or the second angle can be a multiple of the first angle.

The stereoscopic camera module comprises in particular the stereoscopic image recording unit. The image recording unit is in particular received in the camera module and thus protected by the camera module and/or by a housing of the camera module. The stereoscopic image recording unit is in particular accommodated into the stereoscopic camera module. The stereoscopic image recording unit can comprise a first image recording apparatus and a second image recording apparatus, each of which is sensitive to light within a spectral range. Furthermore, the stereoscopic image recording unit can comprise a third image recording apparatus and/or a fourth image recording apparatus, which, for example, are sensitive to light within a further spectral range. In particular, the stereoscopic image recording unit is a 3-D camera system. The first image recording apparatus and/or the second image recording apparatus may, in particular, comprise sensors and prisms mounted back-to-back. Furthermore, the first image recording apparatus and/or the second image recording apparatus and/or the third image recording apparatus and/or the fourth image recording apparatus may comprise filters and/or an optical system.

Using the first image recording apparatus and the second image recording apparatus, for example, stereoscopic images can be recorded within a first spectral range. Using the third image recording apparatus and/or the fourth image recording apparatus, for example, stereoscopic images can be recorded within a second spectral range. This allows fluorescence stereoscopic images to be recorded using the third image recording apparatus and the fourth image recording apparatus. To enable fluorescence imaging, the image recording apparatuses can comprise additional image sensors that are sensitive to light within different spectral ranges. The first image recording apparatus and the second image recording apparatus can each comprise a first image sensor that is sensitive to light at least predominantly within the first spectral range, which is particularly associated with visible light. This means that it is possible to record images within the visible light wavelength range using the first image sensor. This corresponds, for example, to white light image recording. The third image recording apparatus and the fourth image recording apparatus can each comprise a second image sensor that is sensitive to light at least predominantly within the second spectral range, which is particularly associated with near-infrared light. This means that it is possible to record images within the near-infrared light wavelength range using the second image sensor.

Alternatively, the first and second image recording apparatuses can be sensitive in both the visual and near-infrared ranges and record images, such as fluorescence images, in both wavelength ranges.

In particular, the stereoscopic camera module and/or the stereoscopic image recording unit comprise(s) an input optical system that defines an optical axis. The viewing direction extends in particular along the optical axis.

To ensure easy or smooth rotation of the image recording unit, the image recording unit can be rotatably mounted within the camera module. For example, the stereoscopic image recording unit can, however, also be designed to rotate together with the camera module. In this case, the camera module can be rotatably mounted on the camera receptacle, in particular in the image capturing position.

In particular, the stereoscopic image recording unit can be rotated independently of the image capturing position. This makes it possible to decouple the image rectification process by rotating the stereoscopic image recording unit from the image capturing position and/or the oblique view of the endoscope. In particular, the image recording unit can be rotated regardless of the angle at which the camera module and/or the image recording unit is deflected relative to the longitudinal shaft axis.

In addition, in particular in the image recording position, at least one longitudinal axis of the linking element of the at least one linking element can be arranged at an angle relative to a longitudinal axis of the camera receptacle.

In particular, the first angle can be realized between the longitudinal axis of the camera receptacle and the longitudinal axis of the linking element to which the camera receptacle is connected, in the image capturing position. In particular, an angle can be realized between the longitudinal axes of the linking elements which corresponds to the difference between the second angle and the first angle.

For example, in the image capturing position, the angle between the longitudinal axes of the linking elements can correspond to the angle between the longitudinal axis of the camera receptacle and the longitudinal axis of the linking element to which the camera receptacle is connected. Alternatively, the angle between the longitudinal axes of the linking element can differ from the angle between the longitudinal axis of the camera receptacle and the longitudinal axis of the linking element to which the camera receptacle is connected. In particular, the angle between the longitudinal axes of the linking elements is comparable and/or identical. Alternatively, the angle between the longitudinal axes of the linking elements can also be different. For example, the angle between the linking elements can increase in a proximal direction. In particular, the total angle between the shaft longitudinal axis and the camera receptacle longitudinal axis can be a maximum of 30° and/or 45° and/or 75° and/or 90°.

To realize the image capturing position and/or the viewing direction, in particular a plurality of linking elements which are movable relative to each other, in particular pivotable relative to each other, can be arranged, wherein by pushing the linking elements forward relative to the shaft, mobility of a number of linking elements relative to each other can be released, wherein the image capturing position and/or the viewing direction can be defined by the number of released linking elements.

In addition, the camera receptacle can, in particular, have a chamfer located at an edge of a proximal end of the camera receptacle. This allows, in particular, the deflection or the in particular articulated mobility of the camera receptacle, in particular in relation to the linking element to which the camera receptacle is connected, to be defined. Alternatively and/or additionally, at least one linking element can have a chamfer that is located on an edge of a distal end of the linking element. The chamfer on the edge at the distal end can be in particular facing the camera. In particular, the chamfer on the edge at the distal end of the linking element and the chamfer on the edge at the proximal end of the camera receptacle can lie against each other in the image capturing position. This allows, in particular, the deflection or the articulated mobility of the camera receptacle relative to the linking element to which the camera receptacle is connected, to be defined.

Alternatively or additionally, the at least one linking element can have a chamfer which is arranged on an edge of a proximal end of the linking element. In particular, the chamfer on the edge of the proximal end of the linking element and another linking element and/or a shear- and/or tensile-resistant actuating element can define the deflection or, in particular, the articulated mobility of the at least one linking element. Particularly in the image capturing position, the respective adjacent chamfers of the camera receptacle and/or of the at least one linking element that is pushed out of the shaft can lie against each other.

In order to achieve a simple and user-friendly implementation of the image capturing position and/or to be able to use the largest possible camera module, the camera module can be movably mounted relative to the camera receptacle. In particular, the camera module is mounted translationally and/or in combination translationally and rotationally relative to the camera receptacle. In particular, the camera module can be moved from a first position, in particular the insertion position, to a second position, in particular the image capturing position, relative to the camera receptacle. For example, the first position can be the insertion position of the camera module. Furthermore, the second position in particular can be the image capturing position of the camera module and/or a position that the camera module has with respect to the camera receptacle in the image capturing position and/or a distance that the camera module has with respect to the camera receptacle in the image capturing position. In other words, in the second position, the camera module can be located at least partially next to the camera receptacle, in particular when viewed parallel to a longitudinal axis of the camera receptacle. Furthermore, the camera module can still be rotatably mounted relative to the camera receptacle, in particular in the second position.

Furthermore, the camera module and/or the camera receptacle can have a control surface that defines a sliding movement path of the camera module onto the camera receptacle from the first position to the second position, in order to achieve a simple and user-friendly realization of the image capturing position. The control surfaces are made of a material so that the sliding movement path has good sliding properties. In particular, the sliding movement path can be designed in the form of a sliding bearing. The material from which the control surfaces are formed, and/or the material that the control surfaces have, can include metal, in particular medical-grade stainless steel, plastic or ceramic. The plain bearing can also be provided with a friction-reducing coating.

To make it easy to adjust the image capturing position and to be able to avoid unwanted twisting of the cable, the cable can extend along the longitudinal shaft axis. Alternatively or additionally, the cable can be arranged off-center, particularly with respect to the longitudinal shaft axis so that a deflection around a linking element can be produced by applying tension to the cable. In order for the image capturing position to be easily adjustable, the cable can be positioned off-center with respect to the curved section and on an inner side with respect to the radius of curvature of the curved section.

In order for the image capturing position to be easily adjustable, in particular a fiber light guide can be guided along the longitudinal shaft axis to the camera receptacle.

Alternatively or additionally, in the image capturing position, the camera receptacle and at least one linking element can together form a curved section. Furthermore, the light guide can be arranged off-center with respect to the curved section and on an outside side with respect to a radius of curvature of the curved section.

For example, to reduce contamination between the camera receptacle and the camera module, the camera receptacle can be flush with the camera module on the distal side, in particular in the image capturing position. “Flush” means, in particular, that minor deviations between the distal end of the actuating shaft and the distal end of the camera module are permitted.

To make image rectification simple and/or economical, the camera module can be rotatably mounted in and/or on the camera receptacle in the image capturing position. In particular, the camera module can be rotatably mounted on a proximal side of the camera receptacle. Alternatively or additionally, the image recording unit can be rotatably mounted within the camera module.

For ease of use, the image recording unit can be rotated using a cable, in particular to rectify an image. The cable can, in particular, be a torsion cable and/or a power supply cable. The torsion cable can, for example, be routed directly along the power supply cable and/or reinforce the power supply cable and/or encase the power supply cable. The cable can be connected to an electronics unit, in particular a camera control unit (CCU), or the cable can connect the camera module to the electronics unit.

In order to be able to use motors with lower torque at high rotational speeds, a transmission can be arranged or interposed between a drive for rotating the cable and the cable itself. For example, the cable can be electrically driven to rotate the cable. The cable can also be rotated manually, for example, by the user, for example by a rotary wheel.

In particular, to easily achieve rotation between the cable and the shaft, the cable can be rotatably mounted on an electronic unit to which the cable is connected. Furthermore, this makes it possible to simplify the sealing of the shaft. Furthermore, the cable can also be permanently attached to the electronic unit, and the cable can be rotatable together with the electronic unit. This allows for the installation of economical, integrated assemblies in the endoscope device, which include the cable and electronic unit as well as, if applicable, the camera module.

Furthermore, the endoscope device can have at least one fluid line that runs through the shaft to an outlet in the camera receptacle in order to be able to clean the distal end of the camera receptacle and/or the camera module, in particular by means of a fluid, in particular air, in particular compressed air, and/or gas, in particular sterile gas, and/or water. Alternatively and/or additionally, that at least one fluid line can also be used to aspirate a fluid, in particular compressed air, and/or gas, in particular sterile gas, and/or water and/or blood and/or other bodily fluids, from the distal end of the camera receptacle.

The camera receptacle can have at least one opening for the at least one fluid line. Additionally, the camera receptacle can have in particular at least one opening for an light guide in order to couple out illumination light toward the distal end. In particular, the light guide can be connected to a light-outcoupling surface at the distal end of the camera receptacle. The light-outcoupling surface can, in particular, include an optical system.

For simple and convenient implementation of the image capturing position, the endoscope device can further include a shear- and tensile-resistant actuating element. The actuating element can extend from a proximal end of the shaft to a distal end of the shaft and can be attached to at least one linking element. In particular, by applying thrust to the actuating element, at least one linking element and the camera receptacle can be displaced relative to the shaft in order to release the in particular articulated mobility of the linking elements, and/or the camera receptacle, in particular to release the articulated mobility, between the camera receptacle and the linking element. In particular, by applying tension to the actuating element, the at least one linking element and the camera receptacle can be retracted relative to the shaft in order to lock the in particular articulated mobility of the linking elements and/or the camera receptacle. The actuating element can, for example, be in the form of a rod that connects proximally to the at least one linking element or the link chain and extends through the shaft.

Furthermore, for easier and/or more convenient operation of the endoscope device, a proximal end of the actuating element can be connected to a handle.

For easy connection of the light guide and/or the at least one fluid line, in particular the light guide and/or the at least one fluid line can also be routed through the actuating element and/or the at least one linking element and/or the handle.

To ensure smooth rotation of the cable and/or smooth movement of the shear- and tensile-resistant actuating element, the shaft can have a first channel and a second channel which extend at least partially along the longitudinal shaft axis. In particular, the cable can be routed through the first channel. Furthermore, in particular the shear- and tensile-resistant actuating element and/or at least one linking element can be routed through the second channel.

To ensure a simple and effective seal of the endoscope device, the endoscope device can in particular have a sealing body, wherein the sealing body seals the shaft on the proximal side.

To achieve a durable and/or even tighter seal, the sealing body can have a first chamber and/or a second chamber that extend at least partially along a longitudinal axis of the sealing body. To this end, the cable can be sealed against the first chamber by means of a seal, in particular by means of an O-ring. Preferably, in this case, the cable is rotatably mounted on the electronic unit. Alternatively or additionally, the electronic unit that is connected to the cable can be sealed against the first chamber by means of a seal, in particular by means of an O-ring. In this case, the cable can be connected to the electronic unit for conjoint rotation. This means that, in order to achieve the rotation of the cable, the electronic unit can be rotated along with the cable in this case. Furthermore, a shear- and tensile-resistant actuating element can be sealed against the second chamber by means of a seal, in particular by means of an O-ring. Alternatively or additionally, the cable can be sealed against the shaft by means of a seal, in particular by means of an O-ring.

The invention further comprises an endoscope with an endoscope device as described and defined herein.

The present invention will be described by way of example below with reference to the accompanying figures. The drawings, the description, and the claims contain numerous features in combination. A person skilled in the art will also, expediently, consider the features individually and use them in combination as appropriate in the context of the claims.

If there is more than one example of a particular object, only one of them may be provided with a reference sign in the figures and in the description. The description of this example can be transferred accordingly to the other examples of the object. If objects are named using number words, such as first, second, third object, etc., these are used to name and/or assign objects. Accordingly, for example, a first object and a third object may be included but not a second object. However, a number and/or sequence of objects could also be derived using numerical words.

In the drawings:

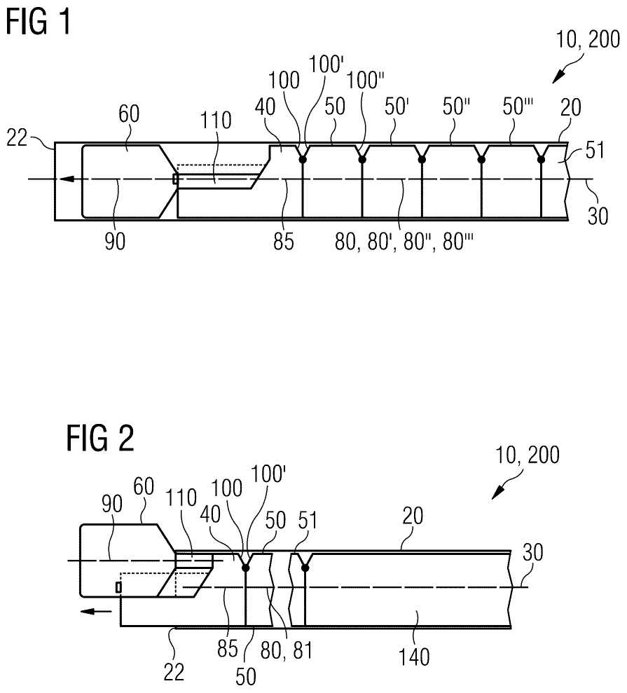

FIG. 1 shows an exemplary embodiment of an endoscope device according to the invention in an insertion position;

FIG. 2 shows an exemplary embodiment of the endoscope device according to the invention, which is shown in FIG. 1, in a configuration between an insertion position and an image capturing position;

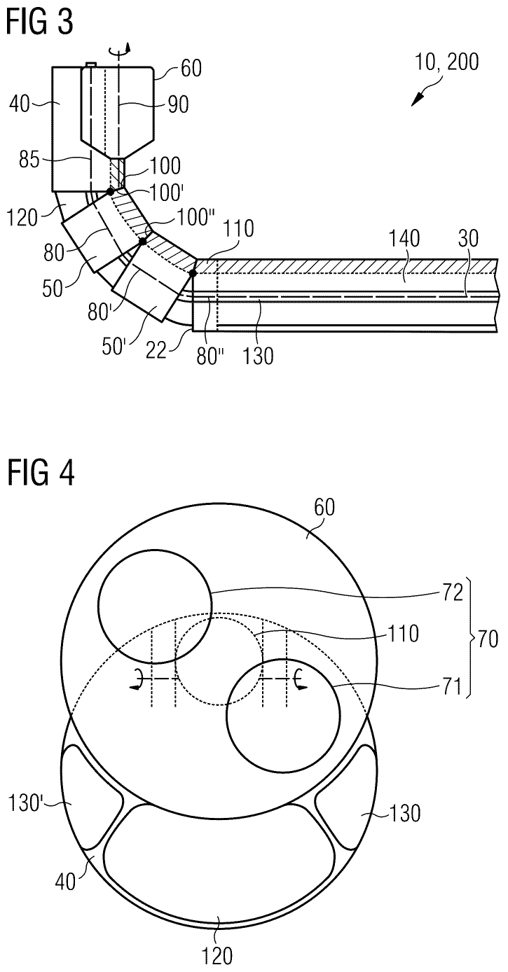

FIG. 3 shows an exemplary embodiment of the endoscope device according to the invention, which is shown in FIGS. 1 and 2, in the image capturing position;

FIG. 4 shows a distal view of an exemplary embodiment of an endoscope device according to the invention;

FIG. 5 shows an exemplary embodiment of an endoscope device according to the invention in an image capturing position;

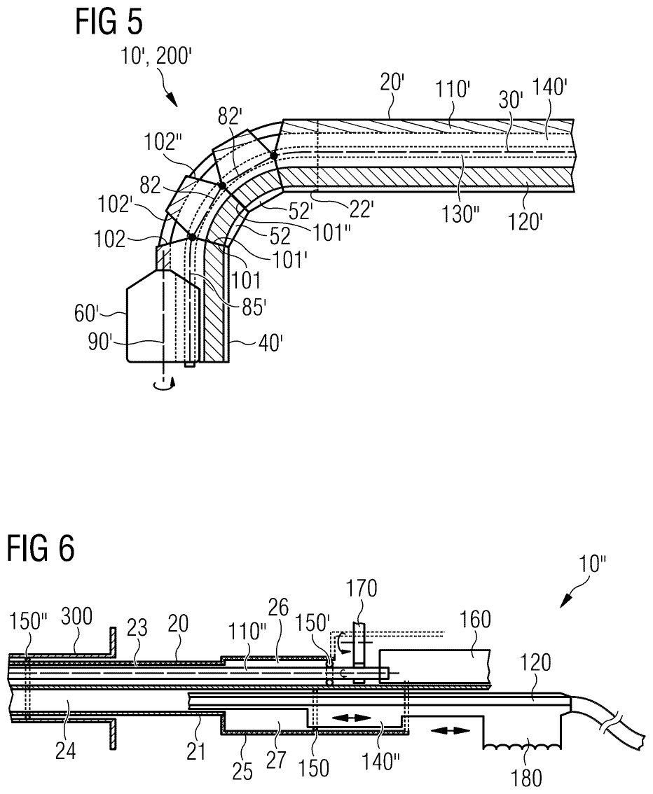

FIG. 6 shows an exemplary embodiment of an endoscope device according to the invention with a shaft sealing body;

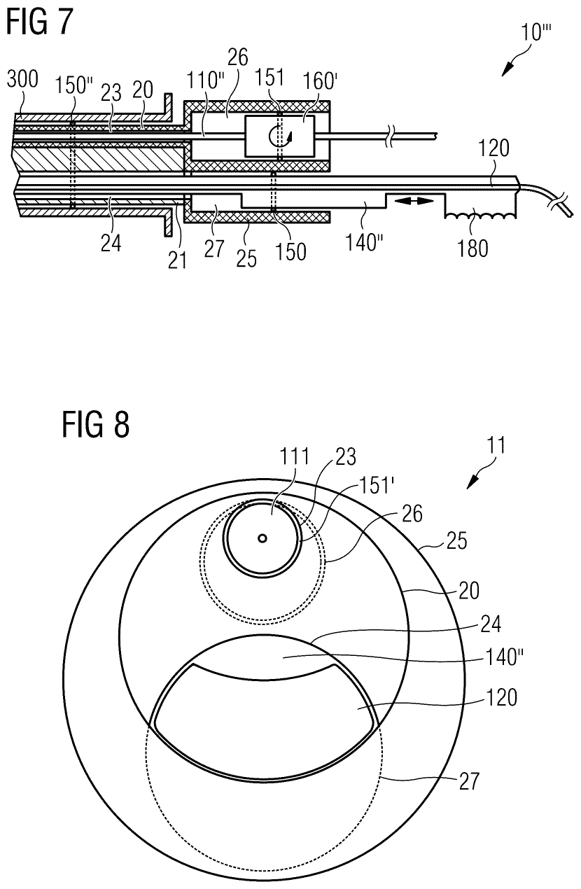

FIG. 7 shows an exemplary embodiment of an endoscope device according to the invention with a shaft sealing body;

FIG. 8 shows an exemplary embodiment of an endoscope device according to the invention with a shaft sealing body;

FIGS. 1 to 3 show an endoscope 200 with an endoscope device 10 according to the invention. The endoscope device 10 has a shaft 20 with a distal end 22, a camera module 60, a camera receptacle 40 and an actuating element 140. The endoscope device 10, shown in FIGS. 1 and 2, has by way of example a plurality of linking elements 50, 50′, 50″, 50′ and 51. The endoscope device 10 shown in FIG. 3 has, by way of example, a reduced number of linking elements, namely the plurality of linking elements 50 and 50′. The endoscope device 10 can however also have only one linking element 50. Furthermore, the camera receptacle 40 can also be arranged directly on the actuating element 140, or the actuating element 140 can form the linking element 50.

In addition, the camera module 60 is connected to a cable 110, in particular a torsion cable and/or power supply cable. The torsion cable can in particular be routed around the supply cable, or the torsion cable can at least partially encase the supply cable. The torsion cable can however also be routed through the supply cable. The camera module 60 and/or an image recording unit that is integrated into the camera module 60 can be rotated through the cable 110 to rectify the image of the image recording unit. The cable 110 can in particular be routed through the camera receptacle 40 and/or the plurality of linking elements 50, 50′, 50″, 50″ and 51. Furthermore, the cable 110, the camera receptacle 40 and/or the plurality of linking elements 50, 50′, 50″, 50′ and 51 can have a slot through which the cable is passed through the shaft. However, the cable 110 can also be routed past camera receptacle 40 and/or the plurality of linking elements 50, 50′, 50″, 50″ and/or 51. Furthermore, the camera receptacle 40 has a chamfer 100 at its proximal end. Furthermore, the plurality of linking elements 50, 50′, 50″, 50″ and 51 each have a chamfer 100′ at their distal end and a chamfer 100″ at their proximal end.

FIG. 1 shows an endoscope device 10 in an insertion position. The camera module 60, the cable 110, the camera receptacle 40 and the plurality of linking elements 50, 50′, 50″, 50″ and 51 as well as the actuating element 140 are arranged inside the shaft 20. In particular, the camera module 60 can also be arranged outside the shaft 20 or at the distal end 22 of the shaft 20 in the insertion position. In the insertion position, a camera longitudinal axis 90, a camera receptacle longitudinal axis 85 as well as a plurality of linking element longitudinal axes 80, 80′, 80″, 80′ and 81 can be aligned parallel, in particular coaxially, to a shaft longitudinal axis 30.

FIG. 2 shows an endoscope device 10 between the insertion position and an image capturing position, or an endoscope device 10 that is in the transition between the insertion position and the image capturing position. The camera module 60, that is shown in FIG. 2, has already been pushed out of the shaft 20 and the camera receptacle 40, that is shown in FIG. 2, has already been pushed out of the shaft 20 at least partially. The camera module 60 has already been pushed onto the camera receptacle 40, thereby moving it in a radial direction relative to the shaft 20 into a position supported on the camera receptacle 40. To ensure smooth movement of the camera module 60 onto the camera receptacle 40, the camera module 60 and/or the camera receptacle 40 can have a control surface that defines a sliding movement path of the camera module 60 onto the camera receptacle 40.

FIG. 3 shows an endoscope device 10 in the image capturing position. In particular, in the image capturing position, the camera longitudinal axis 90 is still arranged parallel to the camera receptacle longitudinal axis 85. Furthermore, the camera longitudinal axis 90 is spaced apart from the camera receptacle longitudinal axis 85. The camera receptacle longitudinal axis 85 and/or the camera longitudinal axis 90 are each arranged at a different angle to the linking element longitudinal axis 80 of the linking element 50, the linking element longitudinal axis 80′ of the linking element 50′ and the linking element longitudinal axis 80″ of the linking element 50″. The linking element 50″ is still accommodated in the shaft 20. That is, the articulated mobility of the linking element 50″ has not yet been released. Therefore, the longitudinal axis 80″ of the linking element 50″ remains parallel, in particular coaxial, to the shaft longitudinal axis 30 of the shaft 20.

As shown, in the image capturing position, the chamfer 100 of the camera receptacle 40 can meet the chamfer 100′ of the linking element 50. Alternatively or additionally, the chamfer 100″ of the linking element 50 can meet a corresponding chamfer of the linking element 50′ as shown. In the endoscope device 10 that is shown in FIG. 3, the articulated mobility of the camera receptacle 40, the linking element 50 and the linking element 50′ is shown by way of example. By way of example, the released articulated mobility of the camera receptacle 40, the linking element 50 and the linking element 50′ in the image capturing position defines a viewing direction which is oriented 90° to the shaft longitudinal axis 30 of the shaft 20. That is, in the exemplary embodiment shown in FIG. 3, the articulated mobility of the camera receptacle 40 and the plurality of linking elements 50, 50′ and 50″ each achieves a rotation of the viewing direction by 30° relative to the longitudinal axis 30 of the shaft 20. The rotation of the viewing direction relative to the shaft longitudinal axis 30 of the shaft 20, which is realized by the articulated mobility of the camera receptacle 40 and the plurality of linking elements 50, 50′ and 50″, can, for example, also be 5°, 10°, 15° and/or 45°. In particular, the rotation is chosen to be large enough that kinking of the cable 110 and/or the at least one fluid line 130 and/or the light guide 120 is prevented.

It is also possible that the articulated mobility of the camera receptacle 40 and the plurality of linking elements 50, 50′ and 50″ allows for different rotations of the viewing direction relative to the shaft longitudinal axis 30 of the shaft 20.

The cable 110 can, for example, be arranged outside the linking element longitudinal axes 80, 80′, 80″ and the camera receptacle longitudinal axis 85, or run along a line that runs outside the linking element longitudinal axes 80, 80′, 80″ and the camera receptacle longitudinal axis 85. In other words, the cable 110 runs off-center with respect to the camera receptacle 40 and/or the linking element 50, 50′ and/or 50″.

Furthermore, the endoscope device 10 can have a light guide 120. The light guide can, for example, be arranged outside the linking element longitudinal axes 80, 80′, 80″ and the camera receptacle longitudinal axis 85, or run along a line that runs outside the linking element longitudinal axes 80, 80′, 80″ and the camera receptacle longitudinal axis 85. In other words, the light guide 120 runs off-center with respect to the camera receptacle 40 and/or the linking element 50, 50′ and/or 50″.

In particular, the light guide 120 and/or the cable 110 can each be arranged on radially opposite sides of the camera receptacle 40 and/or the linking element 50, 50′ and/or 50″. For example, the cable 110 is bent in the image capturing position along an inner radius of the camera receptacle 40 and/or the linking elements 50, 50′ and/or 50″. The light guide 120 can, for example, be bent along an outer radius of the camera receptacle 40 and/or the linking elements 50, 50′ and/or 50″. By bending the light guide 120 along the outer radius of the camera receptacle 40 and/or the linking elements 50, 50′ and/or 50″, the light guide 120 is bent along a smaller radius than if the light guide 120 were bent along the inner radius of the camera receptacle 40 and/or the linking elements 50, 50′ and/or 50″. This can increase the lifespan of the light guide 120. In addition, this can increase the illumination light that is coupled out via the light guide 120 at the distal end of the camera receptacle 40, in particular a light-outcoupling surface of the camera receptacle 40.

FIG. 4 shows a distal view of an exemplary embodiment of an endoscope device according to the invention, with an exemplary distal view of the camera receptacle 40 and an exemplary distal view of a camera module 60. The camera module 60 is connected to the cable 110. The cable 110 is obscured by the camera module in the distal view and is therefore shown with a dashed line. The camera module 60 has an image recording unit 70 with a first image recording apparatus 71 and a second image recording apparatus 72 which are spaced apart from each other at a stereo base distance. The camera receptacle has an opening for the fluid line 130′, an opening for the fluid line 130 as well as the light-outcoupling surface for the light guide 120. The openings for the fluid lines 130 and/or 130′ can be designed in the form of nozzles. In particular, the light-outcoupling surface of the fluid line 120 can be arranged between the openings for the fluid lines 130 and 130′.

FIG. 5 shows another exemplary embodiment of an endoscope device 10′ of an endoscope 200′. The endoscope device 10′ has a shaft 20′ with a distal end 22′, a camera module 60′, a camera receptacle 40′, an actuating element 140 and a plurality of linking elements 52 and 52′. Furthermore, the endoscope device 10′ has a cable 110′, a light guide 120′ as well as at least one fluid line 130″. The cable 110′ is connected to the camera module 60. In particular, the cable 110′ is designed in a manner comparable to cables 110 that are shown in FIGS. 1 to 3. The at least one fluid line 130″ as well as the light guide emerge from the distal end of the camera receptacle, for example, as shown in FIG. 4 for the fluid lines 130 and 130′ and the light guide 120.

The camera receptacle 40′ and the linking elements 52 and 52′ have different chamfers than the camera receptacle 40 and the linking elements 50 and 50′ that are shown in FIG. 3. The camera receptacle 40′ has the chamfers 101 and 102 at a proximal end, which are arranged on opposite edges of the camera receptacle 40′, in particular on opposite sides of a camera receptacle longitudinal axis 85′. Furthermore, the linking element 52 has chamfers 101′ and 102′ at a distal end and chamfers 101″ and 102″ at a proximal end which are each arranged on opposite edges of the linking element 52, in particular on opposite sides of a linking element longitudinal axis 82. The linking element 52′, similar to the linking element 52, also has two chamfers at a distal end and at a proximal end on opposite edges, in particular on opposite sides of a linking element longitudinal axis 82′.

This makes it possible, for example, to rotate the endoscope device 10′ in the direction of the edge of the camera receptacle 40′ which has the chamfer 101, and in the direction of the edges of the linking element 52 which have the chamfers 101′ and 101″. In this case, in particular, in particular if the articulated mobility of the camera receptacle 40′ is released or if the camera receptacle 40′ has been pushed out of the shaft, the edge 101 lies against the edge 101′. To achieve the bend, for example the cable 110′ can be pushed into the shaft 20′ and/or the light guide 120′ can be pulled out of the shaft 20′.

Furthermore, the endoscope device 10′ can also be rotated, for example, in the direction of the edge of the camera receptacle 40′ which has the chamfer 102, and in the direction of the edges of the linking element 52 which have the chamfers 102′ and 102″. In this case, in particular, in particular if the articulated mobility of the camera receptacle 40′ is released or if the camera receptacle 40′ has been pushed out of the shaft, the edge 102 lies against the edge 102′. To achieve the bend, for example the cable 110′ can be pulled out of the shaft 20′ and/or the light guide 120′ can be pushed into the shaft 20′.

FIG. 6 shows an exemplary embodiment of an endoscope device 10″ according to the invention with a shaft sealing body 25 that seals the proximal end of the shaft 20. The shaft sealing body 25 can, for example, be attached to the shaft 20 and/or manufactured together with the shaft 20. In particular, the connection between the shaft sealing body 25 and the shaft 20 is designed fluid-tight.

In particular, the shaft 20 can have a first channel 23 and a second channel 24. Furthermore, the shaft sealing body 25 can have a first chamber 26 and a second chamber 27. In particular, the first chamber 26 is connected to the first channel 23 and the second chamber 27 to the second channel 24. The first chamber 26 and/or the second chamber 27 and/or the first channel 23 and/or the second channel 24 can be designed round and/or semicircular. In particular, the first chamber 26 and/or the second chamber 27 can have a larger diameter than the first channel 23 and/or the second channel 24.

Furthermore, the endoscope device 10″ includes a cable 110″ which is rotatably mounted on an electronic unit 160 and which is guided through the first channel 23 and the first chamber 26. The cable 110″ can be rotated via a transmission 170. For example, the transmission 170 can be connected to an electric motor. Furthermore, a seal 150′, in particular an O-ring, seals the cable 110″ to the first chamber 26.

Furthermore, the endoscope device 10″ includes an actuating element 140″ which is connected to a handle 180. Furthermore, the actuating element 140″ is routed through the second channel 24 and the second chamber 27. Additionally, the endoscope device 10″ has a light guide 120 which is routed through the handle 180 and the actuating element 140″. Furthermore, at least one fluid line can also be routed through the actuating element 140″ and the handle 180. Furthermore, the actuating element 140′ seals against the second chamber 27 by means of a seal 150, in particular an O-ring. Furthermore, a section of the actuating element 140′, on which the seal 150 is arranged, can have a widened cross-section.

As shown, the shaft 20 can be inserted through a trocar 300, for example into the body of a patient. To seal the shaft 20 against the trocar 300, a seal 150″, in particular an O-ring, can be arranged between the trocar 300 and the shaft 20.

FIG. 7 shows an exemplary embodiment of an endoscope device 10′″ according to the invention with the shaft sealing body 25 which seals the proximal end of the shaft 20. In contrast to the endoscope device that is shown in FIG. 6, the exemplary embodiment that is shown in FIG. 7 has a cable 110′″ which is fixed to an electronic unit 160′ for conjoint rotation. That means the cable 110″ is rotated together with the electronics unit 160′. For sealing the first chamber 26, a seal 151, in particular an O-ring, is arranged on the electronic unit 160′ by way of example, which seals the electronic unit 160′ towards the first chamber 26.

FIG. 8 shows an exemplary cross-section of an endoscope device 11 in which the shaft 20 is connected to the shaft sealing body 25. The cross-section runs through a section of the shaft 20. In contrast to and/or in addition to the cable 110″ of the endoscope device 10″ that is shown in FIG. 6 and the cable 110′″ of the endoscope device 10′″ that is shown in FIG. 7, a seal 151′ seals a cable 111 against the first channel 23. The shaft 20 and the shaft sealing body 25 each have a round cross-section, the centers of which are offset from each other or spaced apart from each other. Furthermore, the first channel 23 and the first chamber 26 have a round cross-section, wherein the diameter of the first channel 23 is smaller than the diameter of the first chamber 26. Furthermore, the second chamber is designed rounded to 27. One form of the second channel 24 is formed by the overlap of the second chamber 27 and the shaft 20. In particular, the light guide 120 has a curvature adapted to the round shape of the shaft 20 and at least one curvature that is adapted to the round shape of the second chamber 27.

### LIST OF REFERENCE SIGNS

10, 10′, 10″, 10′″ Endoscope device
11 Endoscope device
20, 20′ Shaft
21 Proximal end of the shaft
22, 22′ Distal end of the shaft
23 First channel
24 Second channel
25, 25′ Shaft sealing body
26 First chamber
27 Second chamber
30, 30′ Longitudinal shaft axis
40, 40′ Camera receptacle
50, 50′, 50″, 50′″ Linking element
51 Linking element
52, 52′ Linking element
60, 60′ Camera module
70 Image recording unit
71 First image recording apparatus
72 Second image recording apparatus
80, 80′, 80″, 80′″ Linking element longitudinal axis
81 Linking element longitudinal axis
82, 82′ Linking element longitudinal axis
85, 85′ Camera receptacle longitudinal axis
90, 90′ Camera longitudinal axis
100, 100′, 100″ Chamfer
101, 101′, 101″ Chamfer
102, 102′, 102″ Chamfer
110, 110′, 110″ Cable
111 Cable
120, 120′ Light guide
130, 130′ Fluid line
140, 140′, 140″ Actuating element
150, 150′, 150″ Seal
151, 151′ Seal
160, 160′ Electronics unit
170 Transmission
180 Handle
200, 200′ Endoscope
300 Trocar

1. An endoscope device, comprising:
a shaft having a shaft longitudinal axis;
a camera receptacle;
at least one linking element to which the camera receptacle is movably attached;
a camera module which has a stereoscopic image recording unit and is configured to accommodated in the camera receptacle,
wherein mobility of the camera receptacle relative to the linking element is released by moving the camera receptacle and the linking element-relative to the shaft, whereby the camera module is deflected from an insertion position to an image capturing position,
wherein, in the image capturing position, the camera module defines a viewing direction which is at an angle to the shaft longitudinal axis, and
wherein, in the image capturing position, the image recording unit is rotatably mounted relative to the actuating shaft.

2. The endoscope device according to claim 1,
wherein the image recording unit is rotatable independently of the image capturing position, the image recording unit is rotatable independently of the angle at which the camera module and/or the image recording unit is deflected relative to the shaft longitudinal axis.

3. The endoscope device according to claim 1,
wherein in the image recording position, at least one linking element longitudinal axis of the at least one linking element is arranged at an angle relative to a camera receptacle longitudinal axis of the camera receptacle.

4. The endoscope device according to claim 1, comprising:
a plurality of linking elements which are arranged so as to be movable relative to each other, wherein by pushing the linking elements forward relative to the shaft, mobility of a number of linking elements relative to each other can be released, wherein the image capturing position can be defined by the number of released linking elements.

5. The endoscope device according to claim 1,
wherein the camera receptacle has a chamfer which is arranged at an edge of a proximal end of the camera receptacle, and/or
wherein the at least one linking element has a chamfer that is arranged on an edge of a distal end of the linking element, and/or has a chamfer that is arranged on an edge of a proximal end of the linking element;
wherein in the image capturing position, the respective adjacent chamfers of the camera receptacle and/or of the at least one linking element which are pushed out of the shaft lie against each other.

6. The endoscope device according to claim 1,
wherein the camera module is movably mounted relative to the camera receptacle,
wherein the camera module is movable from a first position to a second position relative to the camera receptacle, and
wherein the camera module in the second position is located at least partially next to the camera receptacle when viewed parallel to a camera receptacle longitudinal axis of the camera receptacle.

7. The endoscope device according to claim 6,
wherein the camera module and/or the camera receptacle has a control surface which defines a sliding movement path of the camera module onto the camera receptacle from the first position to the second position.

8. The endoscope device according to claim 1, further comprising:
a cable that is connected to the camera module and extends along the longitudinal axis of the shaft,
wherein the cable is arranged off-center with respect to the longitudinal shaft axis so that a deflection around the linking element can be produced by applying tension to the cable.

9. An endoscope device according to claim 1, further comprising:
a light guide which is guided along the longitudinal shaft axis to the camera receptacle,
wherein in the image capturing position, the camera receptacle and the at least one linking element together form a curved section, and wherein the light guide is arranged off-center with respect to the curved section and on an outside side with respect to a radius of curvature of the curved section.

10. The endoscope device according to claim 8,
wherein the cable is arranged off-center with respect to the curved section and on an inside side with respect to the radius of curvature of the curved section.

11. The endoscope device-according to claim 1,
in the image capturing position, the camera receptacle is flush with the camera module on the distal side.

12. The endoscope device according to claim 1,
wherein in the image capturing position, the camera receptacle rotatably supports the camera module, and/or the image recording unit is rotatably supported within the camera module.

13. The endoscope device according to claim 1,
wherein the image recording unit is rotatable by means of a cable, a torsion cable and/or a power supply cable, in order to rectify an image.

14. The endoscope device according to claim 13,
wherein a transmission is arranged between a drive for rotating the cable and the cable.

15. The endoscope device according to claim 13,
wherein the cable is rotatably mounted on an electronic unit to which the cable is connected.

16. The endoscope device according to claim 13,
wherein the cable is permanently connected to an electronic unit, and the cable is rotatable together with the electronic unit.

17. The endoscope device according to claim 1, comprising:
at least one fluid line which runs through the shaft to an outlet in the camera receptacle.

18. The endoscope device according to claim 1, comprising:
a shear- and tensile-resistant actuating element extending from a proximal end of the shaft to a distal end of the shaft and to which at least one linking element is attached,
wherein, by applying thrust to the actuating element, the at least one linking element and the camera receptacle are displaceable relative to the shaft in order to release mobility between the linking elements and/or to release movement between the camera receptacle and the linking element, and
wherein by applying tension to the actuating element, the at least one linking element(s) and the camera receptacle can be retracted relative to the shaft in order to lock the mobility of the linking elements and/or the camera receptacle.

19. The endoscope device according to claim 18,
wherein a proximal end of the actuating element is connected to a handle.

20. The endoscope device according to claim 18,
wherein a light guide and/or at least one fluid line is routed through the actuating element and/or the at least one linking element and/or the handle.

21. The endoscope device-according to claim 1,
wherein the shaft has a first channel and a second channel which extend at least partially along the longitudinal shaft axis,
wherein a cable is routed through the first channel, and
wherein a shear- and tensile-resistant actuating element and/or at least one linking element is routed through the second channel.

22. The endoscope device according to claim 1, comprising:
a sealing body,
wherein the sealing body seals the shaft on the proximal side.

23. The endoscope device according to claim 22,
wherein the sealing body has a first chamber and/or a second chamber which extend at least partially along a longitudinal axis of the sealing body,
wherein a cable and/or an electronic unit that are connected to the cable is sealed against the first chamber by a seal and/or
wherein a shear- and tensile-resistant actuating element seals against the second chamber by a seal.

24. An endoscope with an endoscope device according to claim 1.
